SEQUENCE LISTING (4 sequences)

SEQ ID NO: 1 - protein, 356 aa, Bovine taurus
AQTPAFNKPKVELHVHLDGAIKPETILYYGRKRGIALPADTPEELQNIIGMDKPLSLPEFLAKFDYYMPAIAGCREAVKRIAYEFVEMKAKDGVVYVEVRYSPHLLANSKVEPIPWNQAEGDLTPDEVVSLVNQGLQEGERDFGVKVRSILCCMRHQPSWSSEVVELCKKYREQTVVAIDLAGDETIEGSSLFPGHVKAYAEAVKSGVHRTVHAGEVGSANVVKEAVDTLKTERLGHGYHTLEDTTLYNRLRQENMHFEVCPWSSYLTGAWKPDTEHPVVRFKNDQVNYSLNTDDPLIFKSTLDTDYQMTKNEMGFTEEEFKRLNINAAKSSFLPEDEKKELLDLLYKAYGMPSPA

SEQ ID NO: 2 - DNA, 1074 bp, Bovine taurus
atggctcagaccccggctttcaacaaaccgaaggtagaactgcacgtacacctggatggtgctatcaaaccggagactatcctgtactatggtcgtaagcgtggcatcgctctgccggctgacactccggaagaactgcagaacatcatcggcatggacaaaccgctgtctctgccggaattcctggctaaattcgactactacatgccggctatcgctggttgccgtgaagcagtcaaacgtatcgcttacgaattcgtagaaatgaaagctaaagatggtgtagtatacgttgaagttcgttactctccgcacctgctggcaaactctaaagttgaaccgatcccgtggaaccaggcagaaggcgatctgactccggatgaagtagtttctctggttaaccagggtctgcaggagggtgaacgcgatttcggcgtaaaagttcgttctatcctgtgctgcatgcgccaccagccgtcttggtcttctgaagttgttgaactgtgcaagaaataccgtgagcagaccgtagttgctatcgatctggcaggtgatgaaaccatcgaaggttcttctctgtttccgggtcacgtaaaggcttatgctgaagctgttaaatctggcgtacaccgtactgtacacgcaggtgaagttggttctgctaacgttgttaaagaagctgttgacaccctgaaaactgaacgcctgggtcacggctaccacaccctggaagacaccaccctgtacaaccgtctgcgtcaggaaaacatgcacttcgaagtttgtccgtggtcctcttacctgactggtgcttggaaaccggacaccgaacacccggttgttcgtttcaaaaacgaccaggtaaactactctctgaacactgacgatccgctgatcttcaaatctaccctggacaccgactaccagatgaccaaaaacgaaatgggtttcactgaagaagaattcaaacgtctgaacatcaacgctgctaagtcctcttttctgccggaagatgagaaaaaagaactgctggacctgctgtacaaggcatacggtatgccgtctccggcttaa

SEQ ID NO: 3 - protein, 362 aa, Homo sapiens
AQTPAFDKPKVELHVHLDGSIKPETILYYGRRRGIALPANTAEGLLNVIGMDKPLTLPDFLAKFDYYMPAIAGCREAIKRIAYEFVEMKAKEGVVYVEVRYSPHLLANSKVEPIPWNQAEGDLTPDEVVALVGQGLQEGERDFGVKARSILCCMRHQPNWSPKVVELCKKYQQQTVVAIDLAGDETIPGSSLLPGHVQAYQEAVKSGIHRTVHAGEVGSAEVVKEAVDILKTERLGHGYHTLEDQALYNRLRQENMHFEICPWSSYLTGAWKPDTEHAVIRLKNDQANYSLNTDDPLIFKSTLDTDYQMTKRDMGFTEEEFKRLNINAAKSSFLPEDEKRELLDLLYKAYGMPPSASAGQNL

SEQ ID NO: 4 - DNA, 1089 bp, Homo sapiens
atggctcagacacccgcatttgataaaccgaaagtggaactgcatgtccacctggatggtagcatcaaaccggaaactatcttatattacggtcggcgtcgtggtattgcgttgccggcaaacacggctgaaggtttgctgaacgtgatcggcatggacaaaccgctgaccttgccggattttttggcgaaatttgattattatatgccggcgattgctggttgccgcgaggcaatcaaacgcatcgcgtatgagtttgttgaaatgaaagcgaaagaaggcgttgtgtatgttgaggtccgttacagtccgcatctgctggctaacagcaaggtagaacctatcccctggaaccaagctgaaggcgatctgacgccggatgaagtggttgctctggtgggtcagggtttacaggagggggagcgcgattttggcgttaaagctcgctctattttatgttgcatgcgccatcagcccaattggtccccgaaagtggttgaactttgtaaaaagtaccaacaacagaccgttgtcgcgattgatttggcaggcgatgaaacaattccaggcagctccctgttgccagggcacgtgcaagcgtaccaagaagcagtgaaaagcggcatccaccggactgtccacgccggcgaggtcggtagcgccgaggttgtgaaagaagccgtggacatcctgaaaaccgagcggctgggccatgggtaccacacactggaggatcaggcattatataaccgcttacgccaggaaaatatgcatttcgaaatttgtccgtggagtagttacttaactggcgcgtggaaaccggataccgaacatgcggttatccgcttaaagaatgatcaagcaaattacagtctgaatacagatgatcccctgattttcaagtctaccctggacacagattatcagatgacgaagcgggatatgggatttacggaagaagaatttaagcgtctcaatatcaatgcggcgaaatcttcatttctgccggaagatgagaaacgtgagttgctggatcttctgtacaaggcctacggtatgccgccgagcgcatcggccgggcagaacctg

(19) United States
(12) Patent Application Publication
(10) Pub. No.: US 2019/0343935 A1
(43) Pub. Date: Nov. 14, 2019

(54) METHODS AND COMPOSITIONS FOR TREATING TUMORS

(71) Applicant: University of Louisville Research Foundation, Inc., Louisville, KY (US)

(72) Inventors: John W. Eaton, Prospect, KY (US); Kavitha Yaddanapudi, Louisville, KY (US)

(21) Appl. No.: 16/084,126

(22) PCT Filed: Mar. 10, 2017
(86) PCT No.: PCT/US2017/021950

§ 371 Date: Sep. 11, 2018

(51) Int. Cl. A61K 38/50; A61K 39/395; A61P 35/00; C07K 16/28; A61K 47/60

(52) U.S. Cl. CPC A61K 38/50; A61K 39/3955; A61P 35/00; A61K 2039/505; C07K 16/2818; A61K 47/60; C12Y 305/04004

(57) ABSTRACT

This disclosure provides for methods of treating tumors.

### TECHNICAL FIELD

This disclosure generally relates to methods and compositions for treating tumors.

### BACKGROUND

Adenosine is a purine nucleoside that includes a molecule of adenine attached to a ribose sugar moiety via a glycosidic bond. Adenosine plays an important role in a number of biochemical processes including, without limitation, energy transfer (e.g., as adenosine triphosphate (ATP) and adenosine diphosphate (ADP)) and signal transduction (e.g., cyclic adenosine monophosphate (cAMP)). Adenosine also is a neuromodulator, believed to play a role in promoting sleep and suppressing arousal, and also plays a role in regulating blood flow to organs through vasodilation.

The present disclosure describes the role of adenosine in tumors and, based on that role, provides for methods of treating tumors in a subject. The present disclosure also demonstrates a synergistic effect when immune checkpoint inhibitors (ICIs) are used in combination with the methods described herein.

### SUMMARY

Provided herein are methods for treating tumors.

In one aspect, a method of inhibiting the growth of a tumor and/or reducing the size and/or growth rate of a tumor is provided. Such a method typically includes contacting the tumor with an effective amount of an adenosine deaminase and an effective amount of one or more immune checkpoint inhibitors (ICIs).

In another aspect, a method of treating a subject having a tumor is provided. Such a method typically includes administering an effective amount of an adenosine deaminase to the subject; and administering an effective amount of one or more ICIs.

Representative tumors include, without limitation, an adrenal cancer, a bladder cancer, a bone cancer, a brain tumor, a breast cancer tumor, a cervical cancer tumor, a gastrointestinal carcinoid tumor, a stromal tumor, Kaposi sarcoma, a liver cancer tumor, a small cell lung cancer tumor, non-small cell lung cancer, a carcinoid tumor, a lymphoma tumor, a neuroblastoma, an osteosarcoma, a pancreatic cancer, a pituitary tumor, a retinoblastoma, a basal cell tumor, a squamous cell tumor, a melanoma, thyroid cancer, or a Wilms tumor.

In some embodiments, the adenosine deaminase has at least 80% sequence identity (e.g., at least 90% sequence identity, at least 95% sequence identity, or 100% sequence identity) to SEQ ID NO:1 or 3, wherein the Cys at position 74 has been modified to protect it from oxidation. In some embodiments, the adenosine deaminase is encoded by a nucleic acid having at least 80% sequence identity to SEQ ID NO:2 or 4, wherein the codon encoding the Cys at position 74 has been modified to protect the encoded Cys from oxidation.

In some embodiments, the adenosine deaminase is comprised within a pharmaceutically acceptable carrier. In some embodiments, the adenosine deaminase is PEGylated. In some embodiments, the PEGylated adenosine deaminase is ADAGEN.

In some embodiments, the ICI is selected from the group consisting of Nivolumab (OPDIVO®), Pembrolizumab (KEYTRUDA®), BGB-A317, Atezolizumab, Avelumab, Durvalumab, and Ipilimumab (YERVOY®).

In some embodiments, the contacting or administering step is intratumoral. In some embodiments, the effective amount is an amount that inhibits the growth of the tumor and/or reduces the size and/or growth rate of the tumor without causing toxicity to the subject. In some embodiments, the methods described herein can further include monitoring the tumor for a reduction in size or growth rate.

In some embodiments, the adenosine deaminase and the at least one ICI are combined prior to the administering step. In some embodiments, the adenosine deaminase and the at least one ICI are administered sequentially.

In still another aspect, a method of depleting intratumoral adenosine from a tumor is provided. Such a method typically includes contacting the tumor with an effective amount of an adenosine deaminase. In some embodiments, the tumor is a melanoma or a lung carcinoma. In some embodiments, the administering step is intratumoral and/or intravenous.

In yet another aspect, an article of manufacture is provided that includes an adenosine deaminase and at least one ICI. In some embodiments, the adenosine deaminase and the at least one ICI are each contained within a pharmaceutically acceptable carrier. In some embodiments, the adenosine deaminase and the at least one ICI are contained within a single pharmaceutically acceptable carrier.

In one aspect, a method of depleting intratumoral adenosine from a tumor is provided. Such a method typically includes contacting the tumor with an effective amount of an adenosine deaminase. In another aspect, a method of inhibiting the growth of a tumor and/or reducing the size and/or growth rate of a tumor is provided. Such a method typically includes contacting the tumor with an effective amount of an adenosine deaminase. In still another aspect, a method of treating a subject having a tumor is provided. Such a method typically includes administering an effective amount of an adenosine deaminase to the subject.

In some embodiments, the tumor is selected from the group consisting of a melanoma and a lung carcinoma. In some embodiments, the adenosine deaminase has at least 80% sequence identity (e.g., at least 90% sequence identity, at least 95% sequence identity, 100% sequence identity) to SEQ ID NO:1 or 3, wherein the Cys at position 74 has been modified to protect it from oxidation. In some embodiments, the adenosine deaminase is encoded by a nucleic acid having at least 80% sequence identity to SEQ ID NO:2 or 4, wherein the codon encoding the Cys at position 74 has been modified to protect the encoded Cys from oxidation.

In some embodiments, the adenosine deaminase is comprised within a pharmaceutically acceptable carrier. In some embodiments, the adenosine deaminase is PEGylated. In some embodiments, the PEGylated adenosine deaminase is ADAGEN.

In some embodiments, the contacting or administering step is intratumoral. In some embodiments, the administering step is oral.

In some embodiments, the effective amount is an amount that inhibits the growth of the tumor and/or reduces the size and/or growth rate of the tumor without causing toxI bty to the subject.

In some embodiments, such methods further include monitoring the tumor for a reduction in size or growth rate.

In one aspect, a method of inducing an anti-tumor immune cell response in a subject is provided. Such a method typically includes administering an effective amount of an adenosine deaminase to the subject. Such a method can further include administering a combination of adenosine deaminase and an effective amount of one or more immune checkpoint inhibitors (ICIs) to the patient.

In some embodiments, the anti-tumor immune cell response is an increase in IFN-gamma-producing CD8+ T cells, a decrease in T-regulatory T cells, a decrease in macrophages, an increase in neutrophils, or an increase in dendritic cells.

Unless otherwise defined, all technical and scientific terms used herein have the same meaning as commonly understood by one of ordinary skill in the art to which the methods and compositions of matter belong. Although methods and materials similar or equivalent to those described herein can be used in the practice or testing of the methods and compositions of matter, suitable methods and materials are described below. In addition, the materials, methods, and examples are illustrative only and not intended to be limiting. All publications, patent applications, patents, and other references mentioned herein are incorporated by reference in their entirety.

### DESCRIPTION OF DRAWINGS

FIG. 1 is a graph showing the changed in tumor volume in the presence and absence of ADAGEN. C57BL/6 mice (n=10 mice) were injected subcutaneously with 1×10⁵ Lewis lung carcinoma (LLC) cells (syngeneic to C57BL/6 mice). ADAGEN (2 Units per mouse) was injected intraperitoneally on day 0 and then every other day. Mice in the control group were injected with PBS (vehicle control; n=10 mice). Tumor volume was measured every other day using digital calipers (in mm). * * * , p<0.001; relative to control group.

FIG. 2 are photographs showing tumors in mice that were treated with ADAGEN. C57BL/6 mice (n=3 mice) were injected subcutaneously with 1×10⁵ B16-F10 melanoma cells (syngeneic to C57BL/6 mice). ADAGEN (2 Units per mouse) was injected on day 12 post-tumor cell inoculation and then every day for next 3 days directly into the tumors. Mice in the control group were injected with B16-F10 cells and PBS (vehicle control; n=3 mice). (A) Mouse image showing tumor shrinkage with ADAGEN treatment. (B) Mouse image showing spontaneous vitiligo observed in melanoma-bearing mice after treatment with ADAGEN.

FIG. 3 shows FACS analyses. C57BL/6 mice (n=3 mice) were injected subcutaneously with 1×10⁵ B16-F10 melanoma cells (syngeneic to C57BL/6 mice). ADAGEN (2 Units per mouse) was injected on day 12 post-tumor cell inoculation and then every day for next 3 days directly into the tumors. Mice in the control group were injected with PBS (vehicle control; n=3 mice). Tumor samples were collected from vehicle-treated and ADAGEN-treated mice. Tumors were digested enzymatically and cell suspensions were washed. RBCs were lysed, cells were then resuspended in FACS buffer (2% FBS in PBS) and counted. 1 million live cells from the tumor cell suspensions were blocked with Fc block and tumor infiltrating immune cells were stained with the antibodies specified for T regulatory cells (A), cytokines (B), or myeloid cells (C). Intracellular staining for the quantification of T regulatory cells was done using the FOXP3 staining kit (ebioscience). For cytokine analysis, 1 million tumor cells were re-stimulated with PMA/Ionomycin for 6 hours in the presence of Golgi plug in 24 well plates. Cells were subsequently harvested and stained for the indicated cell surface and intracellular cytokine markers. Samples were acquired on a FACSCanto machine (BD Biosciences).

FIG. 4 is a schematic showing the treatment regimen for combination therapy with PEG-ADA and an immune checkpoint inhibitor.

FIG. 5 is a graph showing the results from therapy with PEG-ADA, an immune checkpoint inhibitor, and a combination thereof.

### DETAILED DESCRIPTION

Tumor-derived secreted and cell surface effectors elicit immunosuppressive signals, resulting in increased T regulatory (Treg) lymphocytes among other suppressive mediators. T regulatory cells have been proposed to contribute to creating a suppressive milieu that protects tumor cells from immune destruction. Although these effector pathways have been the focus of drugs designed to break immune tolerance in late stage cancer patients, immunotherapeutic strategies have largely failed to improve overall survival in cancer patients. Methods and compositions are described herein that can improve current immunotherapeutic strategies, particularly when used in combination.

### Adenosine Deaminase

Adenosine deaminase, also known as adenosine aminohydrolase or ADA, is an enzyme involved in purine metabolism. Adenosine deaminase is assigned to Enzyme Classification (EC) 3.5.4.4, and is responsible for the conversion of adenosine to inosine. As described herein, a tumor can be contacted with an effective amount of an adenosine deaminase, which depletes intratumoral adenosine and, in turn, inhibits growth of the tumor and/or reduces the size and/or growth rate of the tumor. For example, a tumor in a subject can be treated by administering an effective amount of an adenosine deaminase.

As used herein, an effective amount of an adenosine deaminase is an amount that shows significant anti-tumor efficacy but does not result in any adverse events greater than grade 3 (e.g., toxicity in the form of immune related adverse events (irAEs) and/or autoimmune pathologies). For example, an effective amount of an adenosine deaminase can be, for example, 10 U adenosine deaminase/kg of weight of the subject (“10 U/kg”), 20 U/kg, or 30 U/kg).

The amino acid sequence of an adenosine deaminase from bovine is shown in SEQ ID NO: 1 and the amino acid sequence of an adenosine deaminase from human is shown in SEQ ID NO: 3. Representative nucleic acid molecules encoding the bovine and human adenosine deaminase (e.g., codon optimized for expression in E. coli) are shown in SEQ ID NO: 2 and SEQ ID NO: 4, respectively. It would be understood, however, that an adenosine deaminase can be used that has a sequence that differs from either the bovine adenosine deaminase or the human adenosine deaminase (e.g., SEQ ID NOs: 1 or 3 or SEQ ID NOs: 2 or 4, respectively). For example, adenosine deaminase polypeptides and nucleic acids that differ in sequence from SEQ ID NOs: 1 or 3 and SEQ ID NOs: 2 or 4, respectively, can have at least 50% sequence identity (e.g., at least 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% sequence identity) to SEQ ID NOs: 1 or 3 or SEQ ID NOs: 2 or 4, respectively.

In calculating percent sequence identity, two sequences are aligned and the number of identical matches of nucleotides or amino acid residues between the two sequences is determined. The number of identical matches is divided by the length of the aligned region (i.e., the number of aligned nucleotides or amino acid residues) and multiplied by 100 to arrive at a percent sequence identity value. It will be appreciated that the length of the aligned region can be a portion of one or both sequences up to the full-length size of the shortest sequence. It also will be appreciated that a single sequence can align with more than one other sequence and hence, can have different percent sequence identity values over each aligned region.

The alignment of two or more sequences to determine percent sequence identity can be performed using the computer program ClustalW and default parameters, which allows alignments of nucleic acid or polypeptide sequences to be carried out across their entire length (global alignment). Chenna et al., 2003, Nucleic Acids Res., 31 (13):3497-500. ClustalW calculates the best match between a query and one or more subject sequences, and aligns them so that identities, similarities and differences can be determined. Gaps of one or more residues can be inserted into a query sequence, a subject sequence, or both, to maximize sequence alignments. For fast pairwise alignment of nucleic acid sequences, the default parameters can be used (i.e., word size: 2; window size: 4; scoring method: percentage; number of top diagonals: 4; and gap penalty: 5); for an alignment of multiple nucleic acid sequences, the following parameters can be used: gap opening penalty: 10.0; gap extension penalty: 5.0; and weight transitions: yes. For fast pairwise alignment of polypeptide sequences, the following parameters can be used: word size: 1; window size: 5; scoring method: percentage; number of top diagonals: 5; and gap penalty: 3. For multiple alignment of polypeptide sequences, the following parameters can be used: weight matrix: blosum; gap opening penalty: 10.0; gap extension penalty: 0.05; hydrophilic gaps: on; hydrophilic residues: Gly, Pro, Ser, Asn, Asp, Gln, Glu, Arg, and Lys; and residue-specific gap penalties: on. ClustalW can be run, for example, at the Baylor College of Medicine Search Launcher website or at the European Bioinformatics Institute website on the World Wide Web.

Changes can be introduced into a nucleic acid molecule (e.g., SEQ ID NOs: 1 or 3), thereby leading to changes in the amino acid sequence of the encoded polypeptide (e.g., SEQ ID NOs: 2 or 4). For example, changes can be introduced into nucleic acid coding sequences using mutagenesis (e.g., site-directed mutagenesis, PCR-mediated mutagenesis) or by chemically synthesizing a nucleic acid molecule having such changes. Such nucleic acid changes can lead to conservative and/or non-conservative amino acid substitutions at one or more amino acid residues. A “conservative amino acid substitution” is one in which one amino acid residue is replaced with a different amino acid residue having a similar side chain (see, for example, Dayhoff et al. (1978, in Atlas of Protein Sequence and Structure, 5 (Suppl. 3):345-352), which provides frequency tables for amino acid substitutions), and a non-conservative substitution is one in which an amino acid residue is replaced with an amino acid residue that does not have a similar side chain.

As discussed in U.S. Pat. Nos. 8,071,741 and 8,741,283, the Cys at position 74, which is present in both the bovine and human adenosine deaminase sequence, can be oxidized when exposed to a solvent. Therefore, the Cys at position 74 often is changed to a non-oxidizable residue (e.g., Ser) or capped (e.g., with oxidized glutathione) to protect it from oxidation. In addition, adenosine deaminases used in the methods herein may contain one or more polymorphisms or mutations (e.g., lysine at position 198 replaced with glutamine; threonine at position 245 replaced with alanine; and/or glycine at position 351 replaced with arginine). In some instances, an adenosine deaminase sequence can be codon-optimized for a particular organism. Such polymorphic, mutant or codon-optimized sequences typically have a very high sequence identity to a wild type adenosine deaminase (e.g., at least 95%, 96%, 97%, 98%, or 99% sequence identity to SEQ ID NOs: 1, 2, 3 or 4).

Adenosine deaminase adopts a (beta/alpha)₈ barrel structure, and requires a single, bound, divalent cation (zinc or cobalt) in the catalytic pocket for activity. The amino acid residues around the active site are highly conserved in mammals; for example, human and bovine adenosine deaminases are 93% identical. In higher eukaryotes, two different isozymes are encoded by different genes. In humans, ADA1 is a single-chain Zn-binding protein and is the predominant protein; ADA2 is thought to be produced by monocytes and is found in very small quantities. Knockout mutations in ADA1 cause immunodeficiency, whereas mutations that cause overexpression result in hemolytic anemia.

PEGylation is well known in the art, and describes a process by which polyethylene glycol (PEG) polymer chains are attached, either or both covalently and non-covalently, to a molecule or macrostructure such as a drug or a therapeutic protein. PEGylation is achieved by incubating reactive PEG molecules with the molecule. Simply by way of example, see U.S. Pat. Nos. 5,122,614; 5,324,844; 5,612,460; 5,808,096; and 5,349,001. A PEGylated drug or therapeutic protein typically exhibits reduced immunogenicity and antigenicity, as well as increased hydrodynamic size, which can prolong the circulatory time by reducing renal clearance. PEGylation also can improve water solubility. PEGylation of an adenosine deaminase can utilize polymers having a total molecular weight of from about 4,000 Daltons to about 45,000 Daltons.

An adenosine deaminase (e.g., a PEGylated adenosine deaminase) as described herein can be purified from a natural source or recombinantly produced, and provided in a pharmaceutical composition. PEGylated forms of adenosine deaminase, ADAGEN (Pegademase bovine) and ENZ-2279 (see, for example, U.S. Pat. No. 8,071,741; and U.S. Publication Nos. US 2009/0047270 and US 2009/0047271, all of which are incorporated by reference herein in their entirety), which are FDA-approved or in clinical trials to treat severe combined immunodeficiency disease (SCID), can be used in the methods and compositions described herein. Although not wishing to be bound by any particular theory, suppression of the adenosine signaling pathway with adenosine deaminase promotes T cell-mediated anti-tumor immune responses, which can engender effective anti-cancer immunity, particularly against melanomas and lung carcinomas, as described herein.

### Immune Checkpoint Inhibitors

As described herein, a tumor also can be contacted with an effective amount of one or more immune checkpoint inhibitors (ICIs), which, in combination with an adenosine deaminase, can inhibit the growth of the tumor and/or reduce the size and/or growth rate of the tumor synergistically relative to each compound alone. As described herein, a tumor can be successfully treated in a subject by administering an effective amount of one or more ICIs in combination with adenosine deaminase.

Immune checkpoint inhibitors are known in the art as compounds that prevent a host's immune cells from being turned off by cancer cells. Simply by way of example, see, Coffin, 2016, Annals of Oncology, 27 (9):1805-8. Several therapeutic antibodies have been developed against the ligand-receptor interaction between the transmembrane programmed cell death 1 protein (PDCD1, PD-1, or CD279) and its ligand, PD-1 ligand 1 (PD-L1 or CD274). PD-L1 on the cell surface binds to PD1 on an immune cell surface, which inhibits immune cell activity. Therefore, compounds (e.g., therapeutic antibodies) that bind to either PD-1 or PD-L1 and block their interaction can overcome the immune checkpoint and allow the T-cells to attack the tumor.

For example, Nivolumab (OPDIVO®, Bristol-Myers Squibb), Pembrolizumab (KEYTRUDA®, Merck) and BGB-A317 are therapeutic antibodies against PD1, and have been used to treat, with varying degrees of success, melanoma, lung cancer, kidney cancer, Hodgkin's lymphoma, and non-small cell lung cancer, while Atezolizumab, Avelumab and Durvalumab are therapeutic antibodies against PD-L1, and have been used to treat, for example, bladder cancer. In addition, Ipilimumab (YERVOY®, Bristol-Myers Squibb) is a therapeutic antibody that blocks the immune checkpoint molecule, CTLA-4, which is separate from the PD-1/PD-L1 interaction. Ipilimumab has been used in the treatment of melanoma, lung cancer, and pancreatic cancer, in addition to other cancers.

Many ICIs have been approved for use by the FDA, or are in clinical trials. Therefore, the amount that would be considered to be an effective amount for many ICIs are known, are available in the literature, or can be extrapolated therefrom. ICIs typically are administered every two or three weeks for a duration of several weeks through an intravenous infusion for a maximum tolerated dose (e.g., a dose that does not cause any treatment-related adverse events or toxic side effects, e.g., abnormalities in blood counts, liver, renal or cardiac function or electrolytes). In some embodiments, an ICI is administered based on the manufacturer's instructions.

### Methods of Treating a Tumor in a Subject

A subject as used herein typically refers to a human, but also can refer to an animal such as, without limitation, livestock (e.g., cattle, pigs, horses, sheep, turkeys, or chickens), companion animals (e.g., dogs, cats, birds, mice, Guinea pigs, or ferrets), and/or zoo animals (e.g., elephants, lions, giraffes, tigers, or bears).

A tumor as used herein can refer to an adrenal cancer, bladder cancer, bone cancer, a brain tumor, breast cancer, cervical cancer, gastrointestinal carcinoid or stromal tumors, Kaposi sarcoma, liver cancer, lung cancer (e.g., small cell, non-small cell, carcinoid tumor), a lymphoma, neuroblastoma, osteosarcoma, pancreatic cancer, a pituitary tumor, retinoblastoma, skin cancer (e.g., basal and squamous cell, melanoma), thyroid cancer, or a Wilms tumor.

Treating, as used herein, refers to inhibiting the growth of a tumor, reducing the size of a tumor, and/or reducing the growth rate of a tumor. Inhibiting or reducing with respect to a tumor can refer to a reduction in the physical size (e.g., length, width, and/or diameter) of a tumor, in the volume of a tumor, in the number of tumors, in the density of one or more tumors, in the weight or mass of the tumors, or any combination thereof. Inhibiting or reducing with respect to a tumor also can refer to inhibiting or reducing the rate at which a tumor grows (e.g., over a defined period of time relative to the growth rate of the tumor in the absence of (e.g., prior to) treatment with the adenosine deaminase or the adenosine deaminase in combination with an ICI), the rate at which a tumor metastasizes, the rate at which a tumor increases in size, or any combination thereof. In some embodiments, the anti-tumor efficacy of a therapeutic drug can be evaluated by RECIST 1.1 criteria, in which efficacy of a therapeutic drug is assessed in a patient based on Objective Response Rate (ORR), Disease Control Rate (DCR), and Progression Free Survival (PFS) and Overall Survival (OS).

In some embodiments, a “reduction” refers to a decrease (e.g., a statistically significant decrease) in the particular characteristic(s) (e.g., the growth of a tumor, the size of a tumor, and/or the growth rate of a tumor) of at least about 5% up to about 95% (e.g., about 5% to about 10%, about 5% to about 20%, about 5% to about 50%, about 5% to about 75%, about 10% to about 25%, about 10% to about 50%, about 10% to about 90%, about 20% to about 40%, about 20% to about 60%, about 20% to about 80%, about 25% to about 75%, about 50% to about 75%, about 50% to about 85%, about 50% to about 95%, and about 75% to about 95%) relative to the same characteristic(s) in the absence of the treatment (e.g., prior to the treatment, after the treatment, or between treatments) or relative to the same characteristic(s) in a population of subjects having similar tumors (from, e.g., a clinical trial). As used herein, statistical significance refers to a p-value of less than 0.05, e.g., a p-value of less than 0.025 or a p-value of less than 0.01, using an appropriate measure of statistical significance, e.g., a one-tailed two sample t-test.

As described herein, the combination of an adenosine deaminase and at least one ICI exhibits a synergistic inhibitor effect on tumors. For example, the combination of an adenosine deaminase and at least one ICI inhibits the growth of a tumor in a synergistic fashion, reduces the size of a tumor in a synergistic fashion, and/or reduces the growth rate of a tumor in a synergistic fashion. As used herein, “synergy” refers to a combined effect (e.g., of an adenosine deaminase and at least one ICI) that is greater than the additive effect of the adenosine deaminase and the ICI(s) alone.

A pharmaceutical composition as described herein typically is formulated to be compatible with the intended route of administration. As described herein, a pharmaceutical composition including an adenosine deaminase or an adenosine deaminase in combination with an ICI can be administered intratumorally, or a pharmaceutical composition including an adenosine deaminase or an adenosine deaminase in combination with an ICI can be administered parenterally (e.g., intravenously, intramuscularly, subcutaneously, intraperitoneally, intraocularly, intrapleurally, intrathecally, or intrauterine).

In addition to an adenosine deaminase (e.g., a PEGylated adenosine deaminase) and, in some cases, an ICI, a pharmaceutical composition typically includes a pharmaceutically acceptable carrier. As used herein, “pharmaceutically acceptable carrier” is intended to include any and all excipients, solvents, dispersion media, coatings, isotonic and absorption delaying agents, and the like, compatible with administration. Pharmaceutically acceptable carriers for delivering therapeutic compounds (e.g., adenosine deaminase with or without one or more ICIs) are well known in the art. See, for example Remington: The Science and Practice of Pharmacy, University of the Sciences in Philadelphia, Ed., 21^{st} Edition, 2005, Lippincott Williams & Wilkins; and The Pharmacological Basis of Therapeutics, Goodman and Gilman, Eds., 12^{th} Ed., 2001, McGraw-Hill Co.

The type of pharmaceutically acceptable carrier used in a particular formulation can depend on various factors, such as, for example, the physical and chemical properties of the compound, the route of administration, and the manufacturing procedure. Pharmaceutically acceptable carriers are available in the art, and include those listed in various pharmacopoeias. See, for example, the U.S. Pharmacopeia (USP), Japanese Pharmacopoeia (JP), European Pharmacopoeia (EP), and British pharmacopeia (BP); the U.S. Food and Drug Administration (FDA) Center for Drug Evaluation and Research (CDER) publications (e.g., Inactive Ingredient Guide (1996)); and Ash and Ash, Eds. (2002) Handbook of Pharmaceutical Additives, Synapse Information Resources, Inc., Endicott, N.Y.

An adenosine deaminase, alone or in combination with one or more ICIs as described herein, can be administered in an effective amount to a tumor (e.g., to a subject that has a tumor). It would be understood that the adenosine deaminase and the at least one ICI can be combined prior to being administered (i.e., administered in a single composition) or that the adenosine deaminase and the at least one ICI can be administered sequentially. When administered sequentially, it would be understood that the adenosine deaminase can be administered first, or the ICI can be administered first. It would also be understood that, if administered sequentially, the time between when the first component is administered and the time when the second component is administered can be, for example, minutes (e.g., five minutes, ten minutes, fifteen minutes, twenty minutes, thirty minutes, or forty-five minutes), hours (e.g., 1 hour, 2 hours, 8 hours, 12 hours or 18 hours), days (e.g., 1 day, 2 days, 3 days, 5 days, or 7 days) or weeks (e.g., one week, two weeks, three weeks, four weeks, six weeks, or eight weeks) apart. In addition, it would be appreciated that, if administered sequentially, the routes of administration can be different.

Subcutaneous, intramuscular and lymphatic metastases can be injected with the highest tolerated dose of adenosine deaminase (e.g., 3.3 U/kg, 10 U/kg or 30 U/kg of adenosine deaminase) in combination with the highest tolerated dose of at least one ICI (e.g., 2 mg/kg) administered via intravenous infusion every 3 weeks until progression or unacceptable toxicity. The responses obtained in patients undergoing the combination therapy can be compared to patients undergoing ICI monotherapy or adenosine deaminase monotherapy.

Typically, an effective amount is the amount that inhibits the growth of a tumor and/or reduces the size and/or growth rate of a tumor without inducing any adverse effects (e.g., toxicity to the subject). Toxicity and therapeutic efficacy of such compounds, alone or in combination, can be determined by standard pharmaceutical procedures in cell cultures or experimental animals, e.g., by determining the LD₅₀ (the dose lethal to 50% of the population) and the ED₅₀ (the dose therapeutically effective in achieving a 50% response). The dose ratio of toxic to therapeutic effects is the therapeutic index, which can be expressed as the ratio LD₅₀/ED₅₀. An amount that exhibits a high therapeutic index is preferred.

The particular formulation and the effective amount will be dependent upon a variety of factors including, without limitation, the route of administration, the dosage and dosage interval, the sex, age, and weight of the subject being treated, and/or the aggressiveness of the tumor (e.g., the growth rate).

The methods described herein also can include monitoring the tumor. For example, the size of the tumor can be monitored and/or the growth rate of the tumor can be monitored. It would be understood that the size of the tumor can be determined prior to being exposed to an adenosine deaminase and at one or more time points following exposure to an adenosine deaminase. These measurements can be used to monitor the tumor for an inhibition in the growth of the tumor and/or a reduction in the size and/or growth rate of the tumor.

### Articles of Manufacture

This disclosure also provides for articles of manufacture, or kits, that can include one or more adenosine deaminases and one or more ICIs, together with suitable packaging material. As discussed herein, representative adenosine deaminases can be a human adenosine deaminase (e.g., having an amino acid sequence shown in SEQ ID NO:1), a bovine adenosine deaminase (e.g., having an amino acid sequence shown in SEQ ID NO:3), or an adenosine deaminase that has a sequence having at least 50% sequence identity (e.g., e.g., at least 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% sequence identity) to SEQ ID NO:1 or 3. As discussed herein, representative ICIs include, without limitation, Nivolumab (OPDIVO®), Pembrolizumab (KEYTRUDA®), BGB-A317, Atezolizumab, Avelumab, Durvalumab, and Ipilimumab (YERVOY®).

Articles of manufacture provided herein also can include one or more pharmaceutically acceptable carriers and/or one or means for delivering either or both the adenosine deaminase and/or the ICI (e.g., intratumorally or intravenously; e.g., a syringe). Articles of manufacture also can contain a package insert or package label having instructions thereon for administering the adenosine deaminase or the adenosine deaminase in combination with an ICI. Articles of manufacture may additionally include reagents that can be used to, for example, monitor the tumor for an inhibition in the growth of the tumor and/or a reduction in the size and/or growth rate of the tumor.

In accordance with the present invention, there may be employed conventional molecular biology, microbiology, biochemical, and recombinant DNA techniques within the skill of the art. Such techniques are explained fully in the literature. The invention will be further described in the following examples, which do not limit the scope of the methods and compositions of matter described in the claims.

### EXAMPLES

### Example 1

### Administration of ADAGEN Results in a Significant Reduction in Tumor Volume

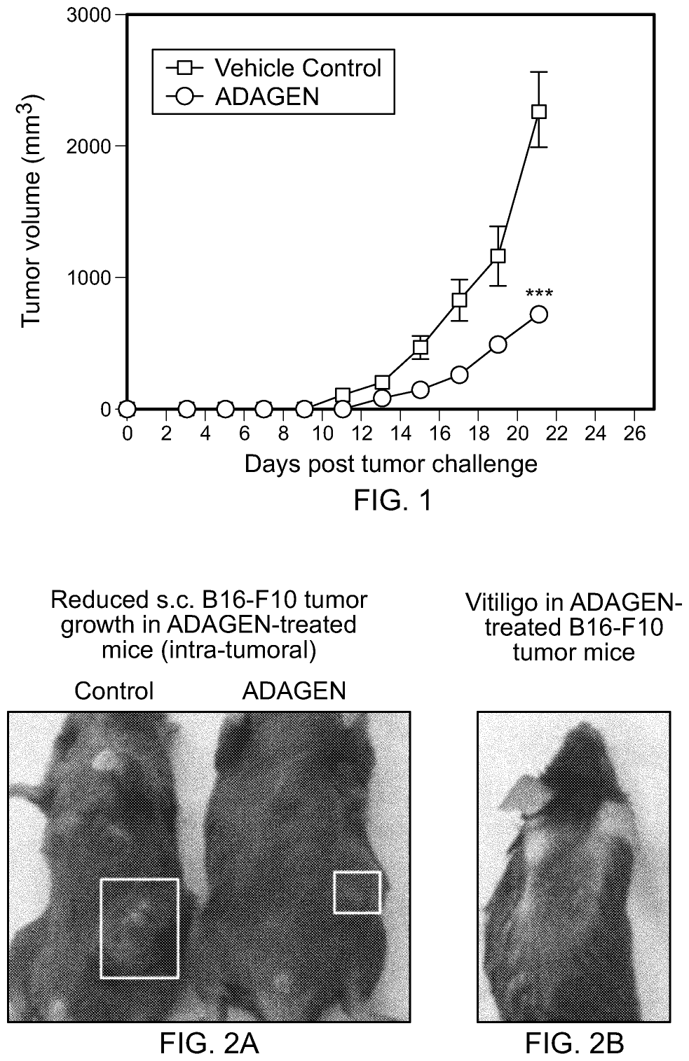

Two syngeneic transplantable mouse tumor models were used; Lewis lung carcinoma (LLC) and B16-F10 melanoma. To assess the anti-tumor activity of ADAGEN (Sigma-Tau Pharmaceuticals, Inc.) against lung cancer, C57BL/6 mice (n=10) were injected subcutaneously with 1×10⁵ LLC cells (syngeneic to C57BL/6 mice). ADAGEN (2 Units per mouse) was injected intraperitoneally on day 0 and then every other day. Mice in the control group were injected with PBS (vehicle control; n=10). Tumor volume was measured every other day using digital calipers. As shown in FIG. 1, LLC tumors in ADAGEN-treated mice were significantly smaller and grew at a much slower rate than those in control mice. These results suggest that systemic treatment with ADAGEN generates potent anti-tumor activity.

### Example 2

### Administration of ADAGEN Results in a Significantly Slower Tumor Growth Rate

Because extracellular adenosine promotes evasion from anti-tumor T cell responses, experiments were designed to determine whether ADAGEN-mediated catabolism of intratumoral adenosine might alter pro-tumorigenic T and myeloid cell responses in tumor bearing mice. B16-F10 murine melanoma cells were injected subcutaneously into syngeneic C57BL/6 mice. Starting at day 12 post-tumor inoculation, when the tumors reached a diameter of >100 mm, 2 units of ADAGEN was injected into the tumors for 4 consecutive days. Control tumors were injected with PBS and tumor outgrowth was followed by caliper measurements. As shown in FIG. 2A, melanomas from ADAGEN injected mice grew at a significantly slower rate than those in vehicle-injected mice. 100% of the ADAGEN treated mice developed depigmentation that progressed to distant locations (FIG. 2B). Clinically, this is called vitiligo and reflects the development of autoimmunity directed against melanocytes. When vitiligo develops in humans with melanoma, it is generally taken as a sign that the melanoma may be immunologically rejected.

### Example 3

### Characterization of Immune Cell Responses in ADAGEN-Treated Tumors

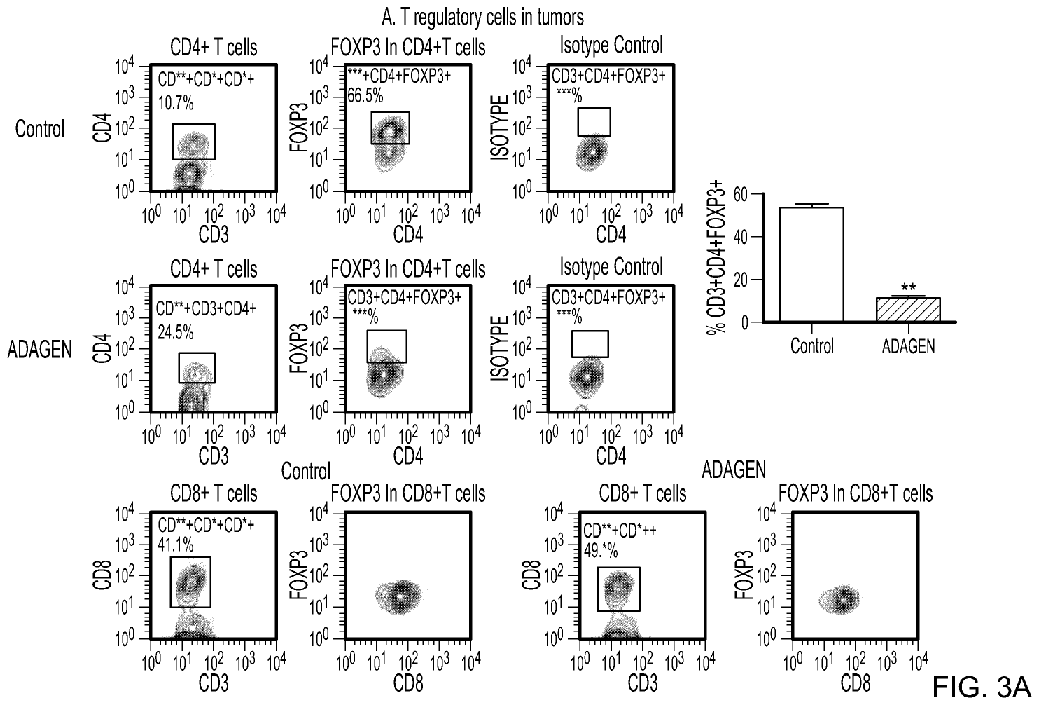

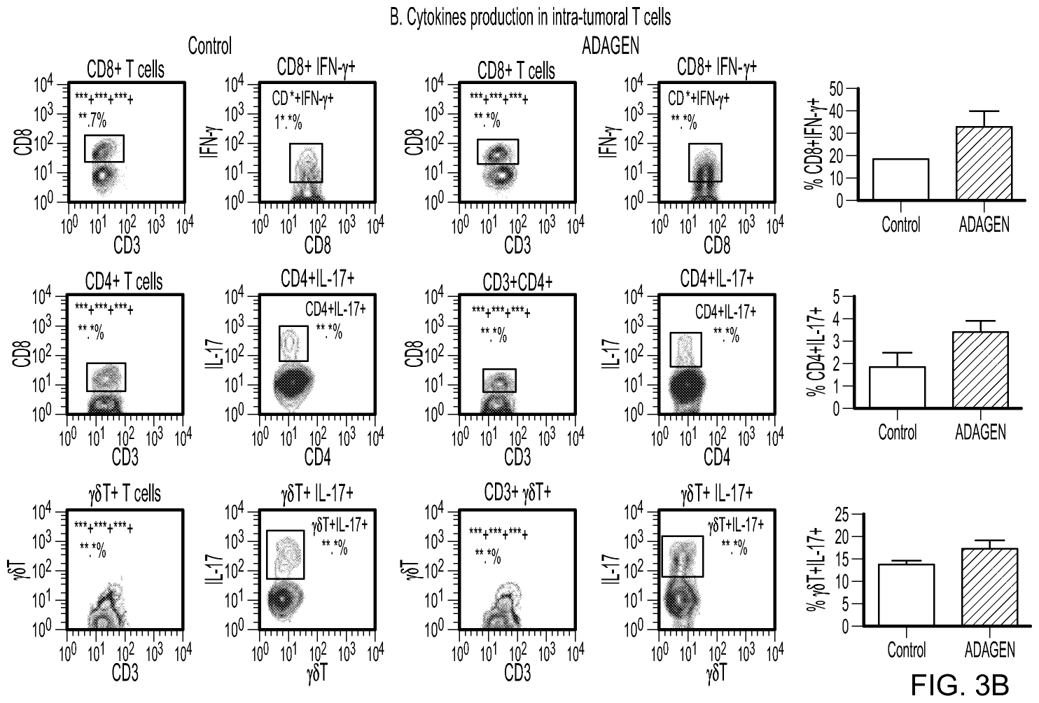

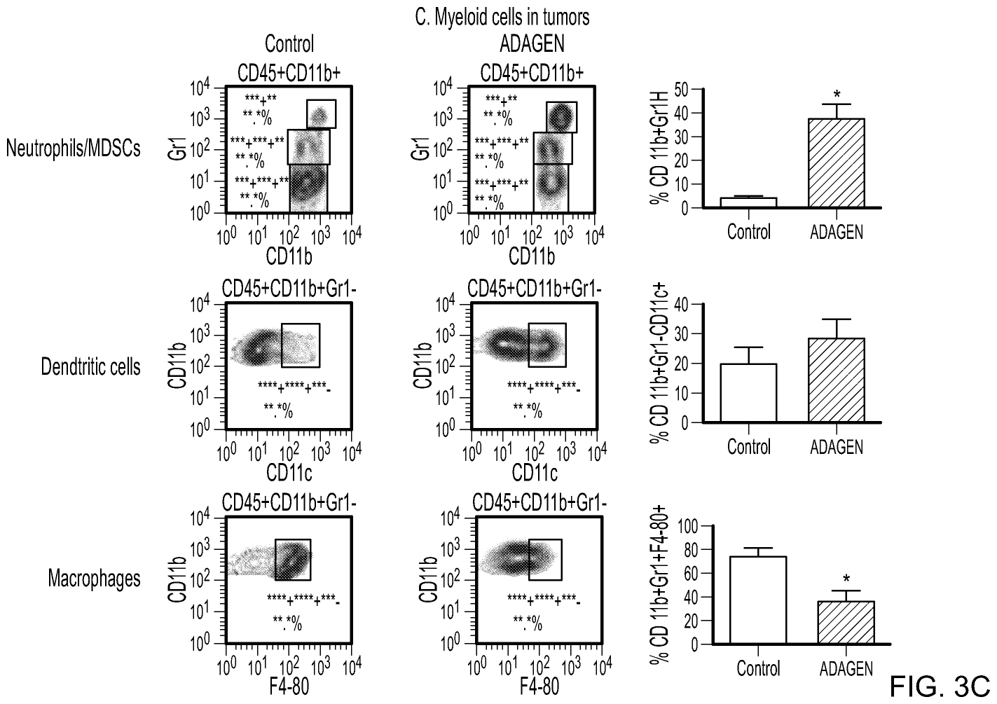

The quantity and quality of anti-tumor immune cell responses generated within the tumors in the ADAGEN-treated and vehicle-treated control mice were assessed. For analysis of tumor-infiltrating immune cells, tumors from ADAGEN-treated and control mice were excised at the end of therapy and analyzed for expression of surface and functional markers of CD4, CD8 T and gamma delta T cells by flow cytometry. Intratumoral T regulatory cells were analyzed using a Foxp3 kit (FIG. 3A). Myeloid cells infiltrating the tumors (dendritic cells, neutrophils and macrophages) were analyzed using surface markers and flow cytometry (FIG. 3C). The data indicate significant increases in IFN-gamma producing CD8+ T cells in ADAGEN-treated tumors compared with the controls (FIG. 3B). More importantly, ADAGEN decreased the frequency of T regulatory CD4+CD25+Foxp3+ T cells and CD11b+Gr1−F4−80+ macrophages while increasing tumor infiltration of anti-tumor immunity inducing CD11b+Gr1hi neutrophils and CD11b+Gr1−CD11c+ dendritic cells within the tumors (FIG. 3).

### Example 4

### Combination Therapy

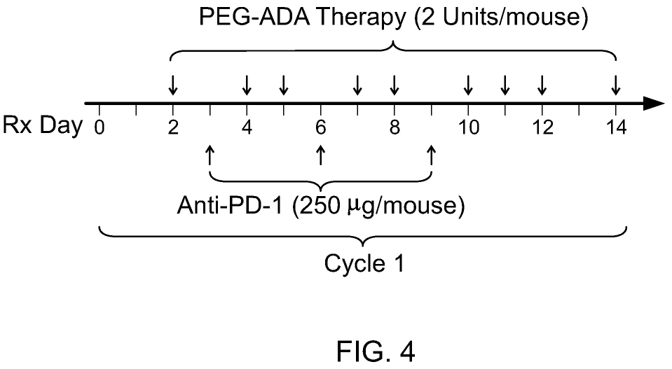

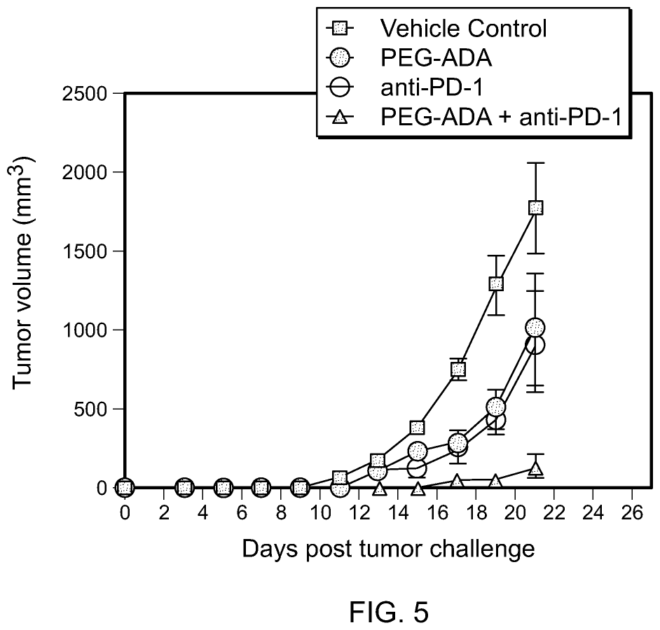

B16-F10 cells were injected at 1×10⁵ cells per mouse subcutaneously. Four groups of eight B16F10-bearing mice/group received the following treatments via the intraperitoneal route using the dosing regimen outlined in FIG. 4: a) Vehicle control; b) anti-PD-1 alone; c) PEG-ADA alone; and d) anti-PD-1 plus PEG-ADA. Tumor sizes were monitored by caliper measurements every other day and plotted as tumor volume per time (FIG. 5).

It is to be understood that, while the methods and compositions of matter have been described herein in conjunction with a number of different aspects, the foregoing description of the various aspects is intended to illustrate and not limit the scope of the methods and compositions of matter. Other aspects, advantages, and modifications are within the scope of the following claims.

Disclosed are methods and compositions that can be used for, can be used in conjunction with, can be used in preparation for, or are products of the disclosed methods and compositions. These and other materials are disclosed herein, and it is understood that combinations, subsets, interactions, groups, etc. of these methods and compositions are disclosed. That is, while specific reference to each various individual and collective combinations and permutations of these compositions and methods may not be explicitly disclosed, each is specifically contemplated and described herein. For example, if a particular composition of matter or a particular method is disclosed and discussed and a number of compositions or methods are discussed, each and every combination and permutation of the compositions and the methods are specifically contemplated unless specifically indicated to the contrary. Likewise, any subset or combination of these is also specifically contemplated and disclosed.

1. A method of inhibiting the growth of a tumor and/or reducing the size and/or growth rate of a tumor in a subject, comprising:
contacting the tumor with an effective amount of an adenosine deaminase and an effective amount of one or more immune checkpoint inhibitors (ICIs).

2. (canceled)

3. The method of claim 1, wherein the tumor is selected from the group consisting of an adrenal cancer, a bladder cancer, a bone cancer, a brain tumor, a breast cancer tumor, a cervical cancer tumor, a gastrointestinal carcinoid tumor, a stromal tumor, Kaposi sarcoma, a liver cancer tumor, a small cell lung cancer tumor, non-small cell lung cancer, a carcinoid tumor, a lymphoma tumor, a neuroblastoma, an osteosarcoma, a pancreatic cancer, a pituitary tumor, a retinoblastoma, a basal cell tumor, a squamous cell tumor, a melanoma, thyroid cancer, or a Wilms tumor.

4. The method of claim 1, wherein the adenosine deaminase has at least 80% sequence identity to SEQ ID NO:1 or 3, wherein the Cys at position 74 has been modified to protect it from oxidation.

5. The method of claim 1, wherein the adenosine deaminase has at least 90% sequence identity to SEQ ID NO:1 or 3, wherein the Cys at position 74 has been modified to protect it from oxidation.

6. The method of claim 1, wherein the adenosine deaminase has at least 95% sequence identity to SEQ ID NO:1 or 3, wherein the Cys at position 74 has been modified to protect it from oxidation.

7. The method of claim 1, wherein the adenosine deaminase has the sequence shown in SEQ ID NO:1 or 3, wherein the Cys at position 74 has been modified to protect it from oxidation.

8. The method of claim 1, wherein the adenosine deaminase is encoded by a nucleic acid having at least 80% sequence identity to SEQ ID NO:2 or 4, wherein the codon encoding the Cys at position 74 has been modified to protect the encoded Cys from oxidation.

9. (canceled)

10. The method of claim 1, wherein the adenosine deaminase is PEGylated.

11. (canceled)

12. The method of claim 1, wherein the ICI is selected from the group consisting of Nivolumab, Pembrolizumab, BGB-A317, Atezolizumab, Avelumab, Durvalumab, and Ipilimumab.

13. The method of claim 1, wherein the contacting step is intratumoral.

14. The method of claim 1, wherein the effective amount is an amount that inhibits the growth of the tumor and/or reduces the size and/or growth rate of the tumor.

15. The method of claim 1, further comprising monitoring the tumor for a reduction in size or growth rate.

16. The method of claim 1, wherein the adenosine deaminase and the at least one ICI are combined prior to the contacting step.

17. The method of claim 1, wherein the tumor is contacted sequentially with adenosine deaminase and the at least one ICI.

18. A method of depleting intratumoral adenosine from a tumor, comprising:
contacting the tumor with an effective amount of an adenosine deaminase.

19. The method of claim 18, wherein the tumor is selected from the group consisting of a melanoma and a lung carcinoma.

20. The method of claim 18, wherein the administering step is intratumoral.

21. An article of manufacture comprising an adenosine deaminase and at least one ICI.

22. The article of manufacture of claim 21, wherein the adenosine deaminase and the at least one ICI are each comprised within a pharmaceutically acceptable carrier.

23. The article of manufacture of claim 21, wherein the adenosine deaminase and the at least one ICI are comprised within a single pharmaceutically acceptable carrier.
